SEQUENCE LISTING (29 sequences)

SEQ ID NO: 1 - protein, 9 aa, Artificial Sequence
  F5-13
TLLLWVLLL

SEQ ID NO: 2 - protein, 9 aa, Artificial Sequence
  F7-15
LLWVLLLWV

SEQ ID NO: 3 - protein, 9 aa, Artificial Sequence
  F33-41
AITTILAAV

SEQ ID NO: 4 - protein, 9 aa, Artificial Sequence
  F105-113
ELDKYKNAV

SEQ ID NO: 5 - protein, 9 aa, Artificial Sequence
  F137-145
FMNYTLNNT

SEQ ID NO: 6 - protein, 9 aa, Artificial Sequence
  F163-171
FLLGVGSAI

SEQ ID NO: 7 - protein, 9 aa, Artificial Sequence
  F182-190
HLEGEVNKI

SEQ ID NO: 8 - protein, 9 aa, Artificial Sequence
  F193-201
ALLSTNKAV

SEQ ID NO: 9 - protein, 9 aa, Artificial Sequence
  F194-202
LLSTNKAVV

SEQ ID NO: 10 - protein, 9 aa, Artificial Sequence
  F196-204
STNKAVVSL

SEQ ID NO: 11 - protein, 9 aa, Artificial Sequence
  F203-211
SLSNGVSVL

SEQ ID NO: 12 - protein, 9 aa, Artificial Sequence
  F210-218
VLTSKVLDL

SEQ ID NO: 13 - protein, 9 aa, Artificial Sequence
  F214-222
KVLDLKNYI

SEQ ID NO: 14 - protein, 9 aa, Artificial Sequence
  F273-281
YMLTNSELL

SEQ ID NO: 15 - protein, 9 aa, Artificial Sequence
  F295-303
KLMSNNVQI

SEQ ID NO: 16 - protein, 9 aa, Artificial Sequence
  F296-304
LMSNNVQIV

SEQ ID NO: 17 - protein, 9 aa, Artificial Sequence
  F417-425
KIMTSKTDV

SEQ ID NO: 18 - protein, 9 aa, Artificial Sequence
  F474-482
SVGNTLYYV

SEQ ID NO: 19 - protein, 9 aa, Artificial Sequence
  F521-529
KINQSLAFI

SEQ ID NO: 20 - protein, 9 aa, Artificial Sequence
  F548-556
IMITTIIIV

SEQ ID NO: 21 - protein, 9 aa, Artificial Sequence
  F553-561
IIIVIIVIL

SEQ ID NO: 22 - protein, 9 aa, Artificial Sequence
  F556-564
VIIVILLSL

SEQ ID NO: 23 - protein, 9 aa, Artificial Sequence
  F559-567
VILLSLIAV

SEQ ID NO: 24 - protein, 9 aa, Artificial Sequence
  F561-569
LLSLIAVGL

SEQ ID NO: 25 - protein, 9 aa, Artificial Sequence
  F563-571
SLIAVGLLL

SEQ ID NO: 26 - protein, 9 aa, Artificial Sequence
  Ebstain-Bar virus peptide GLC-9
GLCTLVAML

SEQ ID NO: 27 - protein, 20 aa, Artificial Sequence
  hepatitis C virus capsid protein peptide HCV C55-74
RPQPRGRRQPIPKARQPEGR

SEQ ID NO: 28 - DNA, 1830 bp, respiratory syncytial virus
acctataaatacggatcagccaccatggagacagacacactcctgctatgggtactgctgctctgggttccaggttccactggtgacggatccatggagctgcccatcctgaaggctaacgctatcaccaccatcctggctgctgtgaccctgtgcttcgcttcctcccagaacatcaccgaggagttctaccagtccacctgctccgctgtgtccaagggttacctgtccgctctgcgtaccggttggtacacctccgtgatcaccatcgagctgtccaacatcaaggagaacaagtgcaacggtaccgacgctaaggtgaagctgatcaagcaggagctggacaagtacaagaacgctgtgaccgagctgcagctgctgatgcagtccacccccgctgctaacaaccgtgctcgtcgtgagctgccccgtttcatgaactacaccctgaacaacaccaagaacaccaacgtgaccctgtccaagaagcgtaagcgtcgtttcctgggtttcctgctgggtgtgggttccgctatcgcttccggtatcgctgtgtccaaggtgctgcacctggagggtgaggtgaacaagatcaagtccgctctgctgtccaccaacaaggctgtggtgtccctgtccaacggtgtgtccgtgctgacctccaaggtgctggacctgaagaactacatcgacaagcagctgctgcccatcgtgaacaagcagtcctgccgtatctccaacatcgagaccgtgatcgagttccagcagaagaacaaccgtctgctggagatcacccgtgagttctccgtgaacgctggtgtgaccacccccgtgtccacctacatgctgaccaactccgagctgctgtccctgatcaacgacatgcccatcaccaacgaccagaagaagctgatgtccaacaacgtgcagatcgtgcgtcagcagtcctactccatcatgtccatcatcaaggaggaggtgctggcttacgtggtgcagctgcccctgtacggtgtgatcgacaccccctgctggaagctgcacacctcccccctgtgcaccaccaacaccaaggagggttccaacatctgcctgacccgtaccgaccgtggttggtactgcgacaacgctggttccgtgtccttcttcccccaggctgagacctgcaaggtgcagtccaaccgtgtgttctgcgacaccatgaactccctgaccctgccctccgaggtgaacctgtgcaacgtggacatcttcaaccccaagtacgactgcaagatcatgacctccaagaccgacgtgtcctcctccgtgatcacctccctgggtgctatcgtgtcctgctacggtaagaccaagtgcaccgcttccaacaagaaccgtggtatcatcaagaccttctccaacggttgcgactacgtgtccaacaagggtgtggacaccgtgtccgtgggtaacaccctgtactacgtgaacaagcaggagggtaagtccctgtacgtgaagggtgagcccatcatcaacttctacgaccccctggtgttcccctccgacgagttcgacgcttccatctcccaggtgaacgagaagatcaaccagtccctggctttcatccgtaagtccgacgagctgctgcacaacgtgaacgctggtaagtccaccaccaacatcatgatcaccaccatcatcatcgtgatcatcgtgatcctgctgtccctgatcgctgtgggtctgctgctgtactgcaaggctcgttccacccccgtgaccctgtccaaggaccagctgtccggtatcaacaacatcgctttctccaacgaattcatcgagggt

SEQ ID NO: 29 - protein, 572 aa, respiratory syncytial virus
MELPILKANAITTILAAVTLCFASSQNITEEFYQSTCSAVSKGYLSALRTGWYVITIELSNIKENKCNGTDAKVKLIKQELDKYKNAVTELQLLMQSTPAANNRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

(19) United States
(12) Patent Application Publication
(10) Pub. No.: US 2010/0172925 A1
(43) Pub. Date: Jul. 8, 2010

(54) HLA-A2-RESTRICTED T-CELL EPITOPES OF THE RESPIRATORY SYNCYTIAL VIRUS FUSION PROTEIN AS PEPTIDE-BASED VACCINES

(75) Inventors: YEN-HUNG CHOW, Miaoli County (TW); Shih-Hen Liu, Taichung (TW); Pele Choi-Sing Chong, Miaoli County (TW)

Correspondence Address: IPC Intellectual Property Connections, INC., 299 Old County Road, Suite 28, San Carlos, CA, 94070, US

(73) Assignee: NATIONAL HEALTH RESEARCH INSTITUTES, Miaoli County (TW)

(21) Appl. No.: 12/648,563

(22) Filed: Dec. 29, 2009

Related U.S. Application Data
(60) Provisional application No. 61/142,376, filed on Jan. 4, 2009.

(51) Int. Cl. A61K 39/12; C07K 7/06; A61K 38/08; A61P 37/04

(52) U.S. Cl. 424/186.1; 530/328; 514/15

(57) ABSTRACT

Respiratory syncytial virus (RSV) fusion protein-specific T-cell epitopes as peptide-based vaccines are disclosed. The isolated peptide contains a human HLA restricted CD8+ T-cell epitope that is specific to RSV F protein. The length of the peptide is no more than 9 or 10 amino acid residues. The peptide may be employed as an immunogen to stimulate cytotoxic T cells, indirectly activate helper T cells type 1, and cause release of cytokines from T cells.

### REFERENCE TO RELATED APPLICATION

The present application claims the priority to U.S. Provisional Application Ser. No. 61/142,376,511, filed Jan. 4, 2009, which is herein incorporated by reference in its entirety.

### FIELD OF THE INVENTION

The present invention relates generally to vaccines against respiratory syncytial virus (RSV), and more specifically to RSV fusion protein (RSV-F) specific T-cell epitopes.

### BACKGROUND OF THE INVENTION

The human respiratory syncytial virus (RSV), a member of the paramyxovirus subfamily Pneumovirinae, is a negative-sense, single-stranded RNA virus of the family Paramyxoviridae. It is the most common cause of viral lower respiratory tract infections in infants and children, affecting about 4 million children globally and leading to about 100,000 hospitalizations and 4,500 deaths per year in the United States alone. RSV infection is associated with recurrent episodes of bronchiolitis, bronchial obstruction and exacerbation of asthma in children. Incidence of RSV infection-induced bronchiolitis has been increasing (WO 03/028759 A1). There is no effective prophylaxis available against RSV infection. Previous attempts to develop a vaccine using formalin-inactivated RSV not only failed but also exacerbated the disease when a subsequent RSV infection occurred (Parrott et al. (1969) “respiratory syncytial virus disease in infants despite prior administration of antigenic inactivated vaccine” Am J Epidemiol 89:422-34). Thus, development of an RSV vaccine has been a high priority at a global level.

Most of the RSV antigens are immunogenic in humans and mice, however, the envelope proteins F and G antigens induce the majority of neutralizing antibodies against RSV. An analysis of the cytotoxic-T-lymphocyte (CTL) repertoire in humans indicated that the N, SH, F, M, M2, and NS2 proteins are strong target antigens. In BALB/c mice, the F, N, and especially the M2 proteins are shown to be the major target antigens of CTL activity (Domachowske et al, (1999) “Respiratory syncytial virus infection: immune response, immunopathogenesis, and treatment” Clin Microbiol Rev 12:298). Virus-specific CTL play a major role in clearance of RSV infection. Both antibodies and MHC-class-1 restricted T lymphocytes (CTLs) mediate protection against RSV infection.

Currently, passive immunization at a monthly interval with a humanized antibody to the RSV-F antigen is considered the only option available to infants, who are at a high risk for developing RSV infection. This approach is inconvenient, expensive, and only partially effective.

Therefore, a previously unaddressed need exists in the art to address the aforementioned deficiencies and inadequacies, especially in connection with development of a safe and effective vaccine against RSV.

### SUMMARY OF THE INVENTION

Identification of virus-specific CD8+ T cell epitope in the study of RSV-induced immunity and vaccine development is important. The protein sequence of the fusion protein (F) of RSV comprising HLA-A*0201 binding motifs was algorithmically analyzed and synthetic peptides were generated. Four out of twenty-five 9-mer peptides, the peptide Nos. 3 (F33-41), 13 (F214-222), 14 (F273-281) and 23 (F559-567), were found to bind to HLA-A*0201 with moderate to high affinity. They were subsequently assessed in T2 cell (which expressing HLA-A*0201)-stabilization assay. They induced a higher frequency of splenic lymphocyte IFN-γ and IL-2 secretion in the ELISAPOT assay from the splenocytes of HLA-A*0201 transgenic B6 (HLA-Tg B6) mice previously immunized with rAd-F0 virus carrying the RSV F gene, or RSV B1 virus. Analysis of the responses revealed that in the HLA-Tg B6 mice previously vaccinated with IFA emulsified—peptides, the peptide number 3, 14, and 23 induced CD8+ lymphocytes activation as determined by an increase in proliferated CD8+ cells and killing activity in immunized spleens. The anti-viral immunity generated by the epitope peptide No. 14 has conferred protection against live RSV-B1 challenge. It reduced the viral load in the lung tissues, presented a faster rate of recovery of body weight loss, as compared to the mice previously immunized with IFA alone. These results show the immunoprotective efficacy of HLA-A*0201-restricted CD8 epitopes for a peptide-based RSV vaccine.

In one aspect, the invention relates to an isolated peptide that comprises a human HLA-A2 restricted T-cell epitope that is derived from the fusion protein of respiratory syncytial virus (RSV-F). The length of the peptide is no more than 10 amino acid residues. In addition, the peptide comprises a HLA-A2 binding motif having a binding affinity to a human HLA-A2 molecule on a cell.

In one embodiment of the invention, the peptide comprises a HLA-A2 binding motif having a binding affinity to a human HLA-A2 molecule on an antigen presenting cell.

In one embodiment of the invention, the aforementioned peptide is a 9-mer selected from the group consisting of SEQ ID NOs: 3, 9, 14, 15, 16, 23 and 24.

In one embodiment of the invention, the aforementioned peptide has a binding affinity at least about 10% higher than that of the hepatitis C virus capsid peptide of SEQ ID NO: 27.

In another embodiment of the invention, the aforementioned peptide possesses the activity of inducing IFN-γ release from an HLA-A2 expressing splenocyte having been previously exposed to RSV. For example, the aforementioned peptide is a 9-mer selected from the group consisting of SEQ ID NOs: 3, 11, 12, 13, 14, 18, 19, 20 and 23.

In another embodiment of the invention, the aforementioned peptide possesses the activity of inducing IL-2 release from an HLA-A2 expressing splenocyte having been previously exposed to RSV. For example, the aforementioned peptide is a 9-mer selected from the group consisting of SEQ ID NOs: 14, 1, 4, 5, 7, 11, 13, 15, 16, 19 and 22.

In another embodiment of the invention, the aforementioned peptide possesses mammalian immunogenicity eliciting CD4+ T cell proliferation. For example, the aforementioned peptide is a 9-mer selected from the group consisting of SEQ ID NOs: 3 and 17.

In another embodiment of the invention, the aforementioned peptide possesses mammalian immunogenicity eliciting CD8+ T cell proliferation. For example, the peptide is a 9-mer selected from the group consisting of SEQ ID NOs: 3 and 14.

Further in another embodiment of the invention, the peptide possesses mammalian immunogenicity eliciting CD8+ T cell proliferation and IFN-γ secretion. Additionally, the peptide may possess mammalian immunogenicity inducing cytotoxic T cell killing responses. For example, the peptide is a 9-mer selected from the group consisting of SEQ ID NOs: 3, 13, 23 and 14.

In another embodiment of the invention, the aforementioned peptide possesses immunoprotective property against RSV infection and is devoid of the sequence of SEQ ID NO: 23.

In another embodiment of the invention, the aforementioned peptide does not induce pulmonary eosinophilia in a mammal.

In another aspect, the invention relates to a composition that comprises a pharmacologically effective amount of one or more than one isolated peptide as aforementioned, and an adjuvant. The adjuvant may be at least one selected from the group consisting of AlPO₄, CFA/IFA, cholera toxin, Escherichia coli heat-labile enterotoxin (LT), liposome, immune-stimulating complex (ISCOM), and immunostimulatory sequences oligodeoxynucleotides (ISS-ODN).

Further in another aspect, the invention relates to a method of providing an immmnoprotective benefit to a human against RSV infection, which comprises the steps of: administering the aforementioned composition to a human in need thereof, thereby providing an immunoprotective benefit to the human against RSV infection. The composition administered to the human for immunoprotection may include the peptide of SEQ ID NOs: 14.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIGS. 1 (a)-(aa) are graphs of flow cytometry spectra showing fluorescence intensity with mean of fluorescence intensity (MFI) ratios of individual synthetic 9-mer peptide Nos. 1 to 25 that bound to HLA-A2 molecules. The term “NC” represents negative control (baseline or background) and “PC” represents positive control.

FIGS. 2A-2B show Th1 cytokines-secreting lymphocytes responsive to HLA-A*0201-restricted RSV F epitope peptides in the RSV or rAd-F0-infected HLA-A*0201 Tg mice.

FIG. 2C is a chart shows characteristics of HLA-A*0201-restricted epitope peptides spanning the RSV F protein.

FIGS. 3A-3C. Epitope-specific CD4+ and CD8+ T-cell activation elicited in the peptide-immunized HLA-A*0201 transgenic B6 mice.

FIG. 4 is a graph showing the CTL activity of the splenocytes of the mice previously immunized with the CD8 epitope peptides as indicated.

FIG. 5 is a graph showing the viral plaque load in the lung tissues of the mice immunized with CD8 epitope of peptides as indicated.

FIG. 6 is a graph showing the change of body weight of the mice post live RSV-B1 challenge.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The human leukocyte antigen system (HLA) refers to the major histocompatibility complex (MHC) in humans. The locus contains many genes that encode cell-surface antigen-presenting proteins. The proteins encoded by certain genes are also known as antigens. The major HLA antigens are HLA class I antigens (A, B and C) and HLA class II antigens (DR, DP and DQ). HLA class I antigens present peptides (9 amino acids) from inside the cell, and the foreign antigens attract CD8 cytotoxic T cells that destroy cells. HLA class II antigens present peptides from outside cells to CD4 T-helper-lymphocytes, which stimulate B-cells.

The term “derived from the fusion protein of respiratory syncytial virus (RSV-F)” shall generally means originated from the RSV-F.

The term “epitope,” also known as antigenic determinant, is the part of a macromolecule that is recognized by the immune system.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Materials and Methods

### Animals and Cell Lines

Human HLA-A*0201-transgenic C57BU6 mice, which expressed human HLA-A*0201 molecules (human class I MHC Ag HLA-A2) on their cells, were a gift from Dr. Liu, the National Health Research Institutes (NHRI), Vaccine R&D Center, Taiwan. BALB/c (H-2K^{d}) and C57BL/6 (H-2K^{b}) mice were purchased from the National Laboratory Animal center. All of mice used in the experiments were six-to-eight-week-old female, maintained in the pathogen-free cages at the Animal Care Center of NHRI throughout the animal study. To examine whether the splenocytes of the mice expressed HLA-A*0201, red blood cell free splenocytes isolated from BALB/c, C57BL/6, and HLA-A*0201 transgenic C57BL/6 mice were added FITC-conjugated anti-human HLA-A*0201 antibody at 4° C. for 1 hour prior to fixation with 1% of formaldehyde. The cells were washed 3 times with ice-cold PBS and analyzed with a flow cytometry. The results indicated that splenocytes isolated from the HLA-A*0201 transgenic C57BL/6 mice expressed HLA-A*0201 molecules, but not the splenocytes isolated from the BALB/c or C57BL/6 mice (data not shown).

Human T2 cells, which express HLA-A*0201 gene but are unable to present endogenous antigen, were purchased from the American Type Culture Collection (ATCC). The cells were cultured in IMDM (Hyclone, Cat. No. SH30228.02)+10% fetal bovine serum (Biological) in an incubator equilibrated with 5% CO₂ at 37° C. Human embryonic kidney cells (293A) and human larynx carcinoma cells (Hep-2) were grown and maintained in DMEM medium (Hyclone) supplemented with 10% fetal bovine serum (Biological), and 1% penicillin/streptomycin (Biological) in 5% CO₂ at 37° C. of incubator.

### Synthesis of HLA-A*0201-Restricted Peptides

Twenty-five HLA-A*0201-restricted 9-mer-peptide sequences (Table 1) from the F protein of RSV-B1 strain were selected by using the computational program of HLA Peptide Binding Predictions developed by BioInformatics and Molecular Analysis Section, National Institutes of Health. The Ebstain-Bar virus peptide, GLC-9 (GLCTLVAML; SEQ ID NO: 26), known to be capable of binding to HLA-A*0201, was used as a positive control in the study of T2 cell-stabilization assay [Colleton et al. (2009) “Primary human immunodeficiency virus type 1-specific CD8+ T-cell responses induced by myeloid dendritic cells”J Virol 83(12):6288-99]. The hepatitis C virus capsid protein peptide RPQPRGRRQPIPKARQPEGR (HCV C55-74; SEQ ID NO: 27), a C57BL/6-specific CD4 epitope, was used as a negative control [Kakimi et al. (1995) “Hepatitis C virus core region: helper T cell epitopes recognized by BALB/c and C57BL/6 mice” J Gen Virol 76 (Pt 5):1205-14]. All of the peptides were synthesized commercially by Echo Chemic, Ltd. and had 75% purity confirmed by high-performance liquid chromatography. Peptides were dissolved in DMSO and their concentrations were adjusted with the culture medium.

TABLE 1

| F peptide | SEQ ID NO: | Amino Acid Sequence |
|---|---|---|
| F5-13 | 1 | TLLLWVLLL |
| F7-15 | 2 | LLWVLLLWV |
| F33-41 | 3 | AITTILAAV |
| F105-113 | 4 | ELDKYKNAV |
| F137-145 | 5 | FMNYTLNNT |
| F163-171 | 6 | FLLGVGSAI |
| F182-190 | 7 | HLEGEVNKI |
| F193-201 | 8 | ALLSTNKAV |
| F194-202 | 9 | LLSTNKAVV |
| F196-204 | 10 | STNKAVVSL |
| F203-211 | 11 | SLSNGVSVL |
| F210-218 | 12 | VLTSKVLDL |
| F214-222 | 13 | KVLDLKNYI |
| F273-281 | 14 | YMLTNSELL |
| F295-303 | 15 | KLMSNNVQI |
| F296-304 | 16 | LMSNNVQIV |
| F417-425 | 17 | KIMTSKTDV |
| F474-482 | 18 | SVGNTLYYV |
| F521-529 | 19 | KINQSLAFI |
| F548-556 | 20 | IMITTIIIV |
| F553-561 | 21 | IIIVIIVIL |
| F556-564 | 22 | VIIVILLSL |
| F559-567 | 23 | VILLSLIAV |
| F561-569 | 24 | LLSLIAVGL |
| F563-571 | 25 | SLIAVGLLL |

### T2 Cell-Stabilization Assay

T2 cells (5×10⁵) cultured in the 96-well U-bottom plates were incubated with 9-mer synthetic peptides (50 μg/mL) and β2-microglobulin (5 mg/mL; Sigma-Aldrich) at 28° C. for 16-18 hours. Brefeldin A (10 μg/mL) was added then and the cells were then incubated at 37° C. for 3 hours. The cells were washed and stained with fluorescence isothiocyanate (FITC)-conjugated mouse anti-human HLA-A2 antibody (SeroTec, Cat. No. MCA2090F). Peptides-loaded T2 cells were run on the FACScan flow cytometer and analyzed by using the CellQuest software (Becton Dickinson Immunocytometry System). The mean fluorescence intensity (MFI) represents the binding activity of each peptide. The background MFI (i.e., negative control) was obtained from the peptide-unloaded T2 cells.

Preparation of RSV-B1 Strain Stocks and rA-F0

Human RSV-B1 strain (VR-1580) was purchased from the ATCC and propagated in Hep-2 cells as previously described [Shao et al. (2009) “Immunogenic properties of RSV-B1 fusion (F) protein gene-encoding recombinant adenoviruses” Vaccine July 19]. Partial purification of the virus was performed by centrifugation of the cell supernatant through a 15% sucrose/PBS gradient for 2 hours at 30,000 rpm. The virus was collected and resuspended in PBS, pH 7.2. The titer of RSV was determined by a standard plaque assay. Briefly, 100 μL of varying dilutions of purified virus preparations were added to Hep-2 cells (5×10⁵/well) cultured in 12-well plates (Corning). Each of the cultures was then overlaid with DMEM containing 1.5% methylcellulose (Sigma-Aldrich) and incubated for 5 to 6 days for the plaques to develop. Plaques stained with hematoxylin and eosin (H/E) were counted under a light microscope. The concentration of the virus was expressed as plaque-forming units per mL (pfu/mL).

The construct rAd-F0, a recombinant Ad5 carrying the RSV-B1 fusion (F) glycoprotein gene, was generated and described previously [Shao et al. ibid.]. Purification and concentration of the rAd-F0 was achieved by ultracentrifugation through a 15% sucrose/PBS gradient at 20,000 rpm for 60 min. The virus was then resuspended in PBS, pH 7.2, and the titer determined by the modified standard plaque assay described above. Various dilutions of rAd-F0 virus were added to 293A cells plated in 6-well tissue culture plates. After overlaying the culture with DMEM containing 0.75% methylcellulose, the culture was incubated at 37° C. for 10 to 12 days and plaques stained with WE were counted. The yield of rAd-F0 was usually around 1×10⁹ pfu/mL.

### Immunization and Live RSV Challenge of Mice

HLA-A2.1 transgenic C57BL/6 mice were anesthetized with isoflurane and primed with 1×10⁷ pfu/50 μL of rAd-F0, rAd-LacZ or 1×10⁴ live RSV B1 via the intranasal (i.n.) route. Twenty days later, animals were boosted i.n. with the same dose of respective immunogens. For peptide-based vaccination, transgenic mice were immunized subcutaneously (s.c.) with 50 μg of individual synthetic peptides emulsified in 100 μL of incomplete Freund's adjuvant (IFA) and then boosted s.c. with the same dose of respective peptides mixed with IFA one week later. Mice were sacrificed 7 days after the boost. The splenocytes were isolated and stimulated with synthetic CD8 epitope peptides in the presence of 5-(6)-carboxyfluorescein diacetate succinimidyl ester (CFSE) for 4 and 8 days. Proliferated CD4⁺ or CD8⁺ T cell counts were analyzed by flow cytometry (BD FACSCalibur) using PE-labeled specific antibody against CD4 or CD8, respectively. Cell dividing index (CDI), a ratio of a portion of CD4 or CD8 positive lymphocytes with low CFSE staining signal (CFSE^{low}) divided by a portion of high CFSE staining signaling (CFSE^{high}) of cells, represents the proliferating lymphocytes responsive to individual epitope peptides. For challenge studies, 1×10⁶ pfu of live RSV-B1 was administered i.n. seven days after the second immunization of peptide vaccines.

### Enzyme-Linked Immunosorbent Spot (ELISPOT) Assay

RBC-free splenocyte (5×10⁶ cells) suspensions from the spleens of individual mice were seeded onto wells of 96-well filtration plates (Millipore) pre-coated with capture monoclonal antibodies for murine IL-2, IL-4 or IFN-γ (0.5 μg per well) purchased from eBioscience and blocked with a conditioned medium (CM) for 1 hour at room temperature. The splenocyte cultures were treated with RSV F 9-mer synthetic peptides (2.0 μg each) or GLC-9 (SEQ ID NO: 26) dissolved in the conditional medium (CM; 100 μL). The splenocyte cultures incubated with Con A (10.0 mg per mL) were used as positive controls. The splenocyte cultures alone were used as negative controls. Experimental cultures were kept in a 37° C. incubator equilibrated with 5.0% CO₂ for 48 hours. The wells of the ELISPOT plates were washed three times with 200 μL of washing buffer (PBS-Tween 20), and added 0.2 μg of the corresponding biotinylated detection monoclonal antibodies specific for IL-2, IL-4 and IFN-γ to detect the binding of capture antibodies to the respective cytokines.

Following 2 hr of incubation at room temperature, the plate was washed with 200 μL of wash buffer. Avidin-HRP (100 μl) diluted at 1:10000 in the assay buffer was added to the wells, and the plates were left at room temperature for 45 minutes. The plates were washed four times with wash buffer, followed by the addition of AEC (3-amine-9-ethylcarbazole, Sigma) substrate (100 μl/well). The substrate was allowed to react for 30 minutes at room temperature in the dark. The plates were washed with water, air-dried overnight, and the spots per culture were scored using the immunospot counting reader (C.T.L. IMMUNOSPOT, CELLULAR TECHNOLOGY LTD). Results were expressed as the number of cytokine-secreting cells per 5×10⁵ splenocytes seeded at culture initiation.

### Cytotoxicity Assay

A non-radioactive assay of cell-mediated cytotoxicity was performed using green fluorescent probes 5-(6)-carboxyfluorescein diacetate succinimidyl ester (CFSE) to label target cells in combination with 7-amino-actinomycin D (7AAD), a red fluorescent probe to label dead effector and dead target cells. The assay was used to assess the presence of the RSV F protein-specific CTLs in the spleens or lungs of the immunized animals [Russell et al. (2002) “Lymphocyte-mediated cytotoxicity” Annu Rev Immunol 20:323-70]. The assay protocol was in accordance with the manufacturer's instructions (Cayman Chemical, Michigan, USA). The assay entailed culturing splenocytes (5×10⁶) with a candidate synthetic peptide (5.0 μg/mL) known to comprise a dominant HLA-A*0201-restricted CTL epitope in the presence of recombinant IL-2 (10 U/mL) at 37° C. for 5 days. Targets (T2 cells) were treated with the synthetic peptide (2.0 μg/mL) for 2 hours at 37° C. prior to the assay. Untreated T2 cells served as a negative control. The peptide-treated target cells (10⁶) were suspended in 1×CFSE staining solution (1.0 mL), incubated at room temperature for 15 minutes, and centrifuged at 400×g for 5 minutes to remove the supernatant.

The CFSE-labeled target cells were resuspended in the culture medium and incubated at 37° C. of CO₂ incubator for 30 minutes. The CFSE-labeled target cells were plated onto 12-well plates (10⁵/well, 10004) and co-cultivated with an appropriate number of in vitro restimulated splenocytes culture (as effectors cells) in defined effector:target (E:T) ratios in triplicate. Four hours later, the cell mixture was centrifuged and the pellet resuspended in the 7AAD staining solution (1.0 mL), and placed in the dark for 15 minutes at 4° C. The control target cells without CFSE or 7-AAD staining or with CFSE staining only were included. The cells were centrifuged and the pellet resuspended in 1.0 mL of assay buffer for analysis with a flow cytometry. The CFSE positive cells were measured in the FL1 channel and 7-AAD stained cells were measured in the FL3 channel. The percentage of CFSE/7-AAD double positive target cells (i.e., T2 cell death) was analyzed.

### Viral Load Determination

Whole lungs from individual experimental mice were removed and homogenized. The homogenized tissues were centrifuged at 3,000 rpm for 10 min at 4° C. to sediment cell debris. Supernatants were collected, serially diluted, and tested for their ability to infect Hep-2 cells in the plaque formation assay.

### Statistical Analysis.

Unpaired, two-tailed Student t test was performed to compare the results obtained from the different experimental groups of mice. Results are considered statistically significant when the p value is <0.05. Symbol * and ** are used to indicate p values <0.05 and <0.01, respectively.

### Results

### Identification of HLA-A2-Restricted CD8 Epitopes of the RSV F Protein

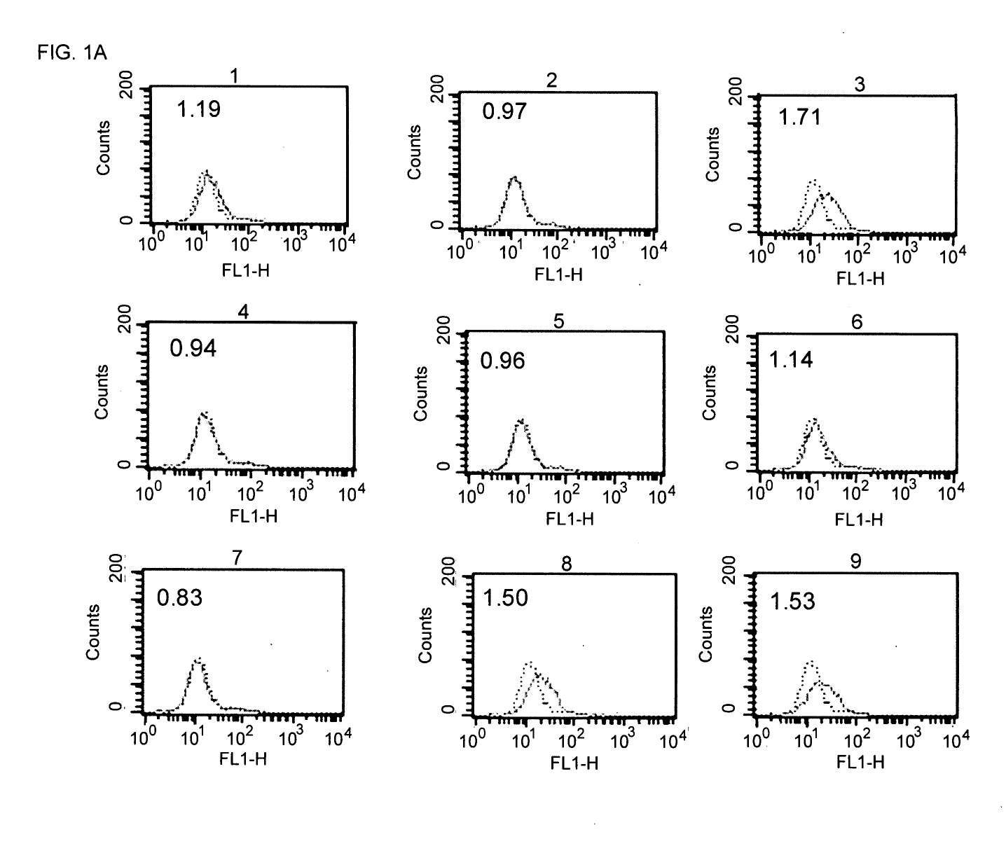

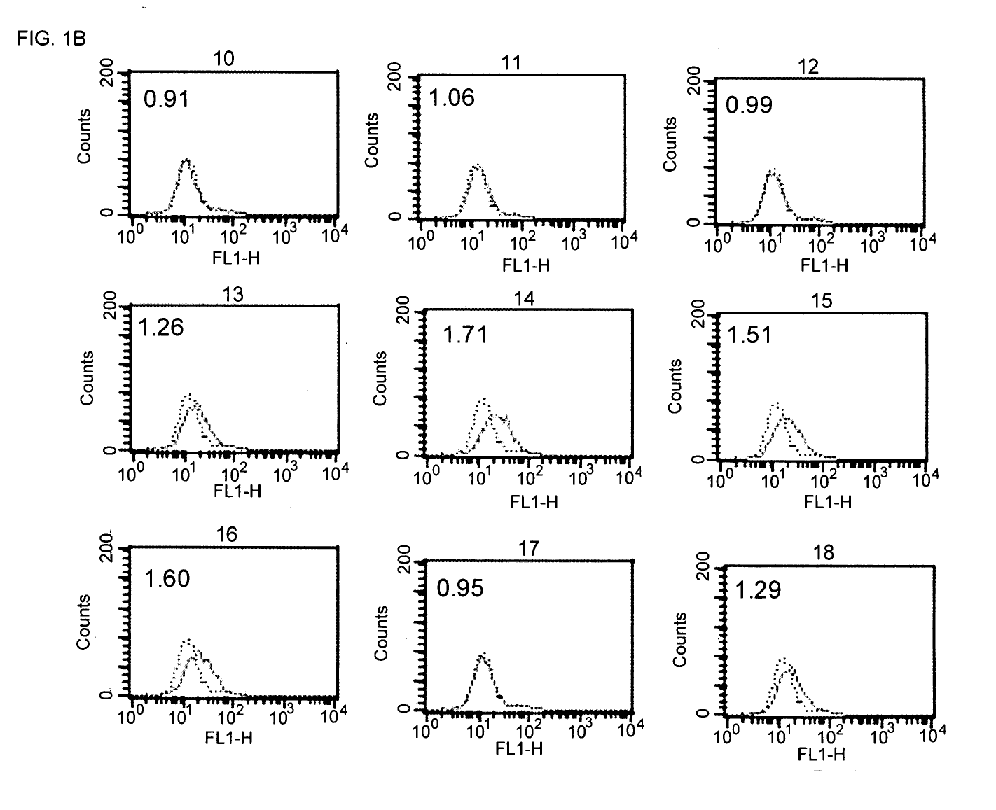

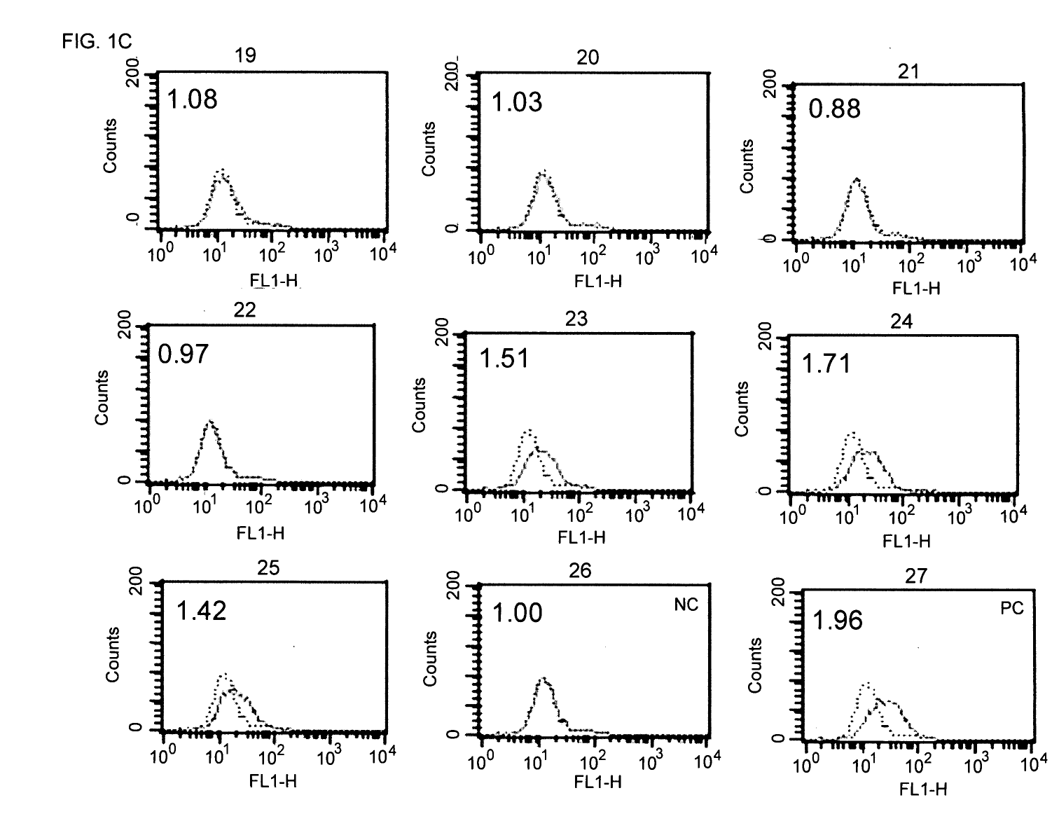

Twenty-five 9-mer synthetic peptides within the RSV F glycoprotein predicted to encompass HLA-A*0201 binding motifs were synthesized (Table 1). Each was tested for its activity in binding HLA-A*0201 molecules using T2-based binding assay. As shown in FIG. 1, the mean of fluorescence intensity (MFI) ratio of the positive control (PC, FIG. 1aa) Epstein-Barr virus peptide (GLC-9, a previously defined HLA-A*0201-restricted CTL epitope) was twofold (MFI=1.96; FIG. 1aa) of the baseline (negative control or NC: FIG. 1z) (MFI=1). Individual epitope peptides from the RSV F exhibited differential MFI ratios of binding affinity to the HLA-A*0201 molecule. The MFI ratios of the peptides Nos. 3, 9, 14, 15, 16, 23, and 24 were about 1.5 fold of the negative control. The peptides Nos. 1, 6, 8, 13, 18, and 25 showed a moderate binding affinity (10% higher than the baseline). The rest of the tested peptides had no binding activity to the HLA-A*0201 molecules.

To screen these peptides for their capability of RSV F-specific T cells activation, spleen lymphocytes from the rAd-F0- or RSV B1 virus-immunized HLA-A2.1 transgenic C57BL/6 (HLA-Tg B6) mice were treated with the individual RSV F peptides at day 10 post the virus infection. The production of IFN-γ and IL-2 was measured by using ELISPOT assays. Splenocytes isolated from the HLA-Tg B6 mice expressed HLA molecules, which had been confirmed by flow cytometry against FITC-conjugated HLA-A*0201-specific antibody (data not shown).

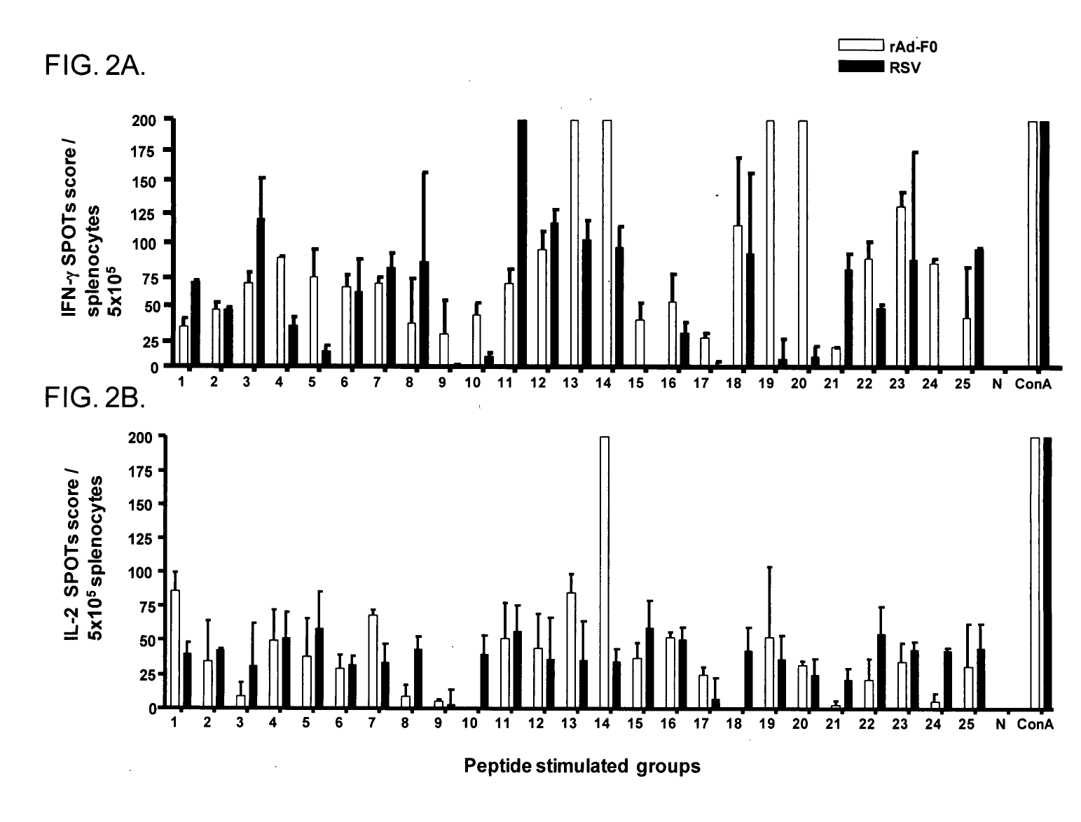

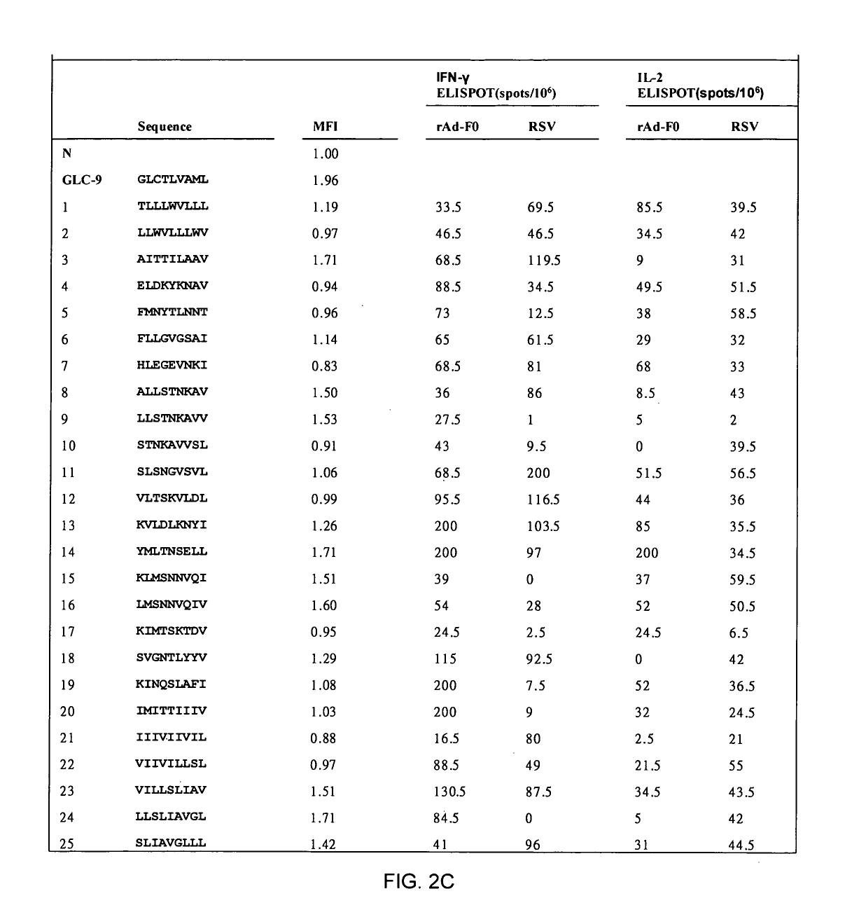

FIGS. 2A-2B show Th1 cytokines-secreting lymphocytes responsive to HLA-A*0201 specific RSV F epitope peptides in RSV or rAd-F0-infected HLA-A*0201 Tg mice. Splenocytes were collected on day 30 from HLA-A*0201 Tg C57BL/6 mice primed and boosted at an interval of 20 days apart via the intranasal route with 10⁷ pfu of rAd-F0 or RSV-B1. They were cultured and supplemented with murine IL-2 in the presence of 20 μg of individual RSV F peptide, or 5 μg/ml Con A for 5 days. Splenocytes (5×10⁵) were seeded onto anti-IFN-γ (A) and IL-2 (B) capture antibody coated wells of an ELISPOT plate for 2 days as described in Materials and Methods. Cytokine-positive immunospots were then developed using the reagents and protocol provided in the assay kit. Results were expressed as number of cytokine immunospots t two standard deviations for each experimental group. Results were expressed as cytokine-secreting spots for each test sample. Bars correspond to mean titers for each experimental group.

The cytokine ELISPOT assay indicated that a higher level of IFN-γ secretion was obtained from either rAd-F0- or RSV B1-immunized mouse splenocytes (over 100 spots) stimulated with peptide Nos. 3, 11, 12, 13, 14, 18, 19, 20, and 23. The peptide 1, 2, 4, 5, 6, 7, 8, 16, 21, 22, 24, and 25 were found to exhibit moderate stimulation of IFN-γ secretion (over 50 spots) (FIG. 2A). Additionally, the individual RSV F peptides expressed various levels of IL-2 production, which could be grouped as higher production peptide, such as peptide No. 14 (over 100 spots), and moderate production peptides, such as peptide Nos. 1, 4, 5, 7, 11, 13, 15, 16, 19, and 22 (over 50 spots) (FIG. 2B). However, a background of IL-4 production was detected in all of the samples (data not shown). In summary, among these CD8 epitope candidates, peptide Nos. 3 (F33-41), 13 (F214-222), 14 (F273-281), and 23 (F559-567) exhibited a strong binding activity with HLA-A*0201 molecule as well as higher Th1 cytokines production (FIG. 2C). They were chosen for the further examination of the immunogenicity of epitope-based vaccination in HLA-Tg B6 mice.

### Induction of Epitope-Specific T Lymphocytes Activation in Peptide-Vaccinated HLA-A *0201 Transgenic B6 Mice

To test the immunogenicity of these peptides, HLA-Tg B6 mice were immunized twice with individual IFA-emulsified peptide No. 3, 14, 23, or peptide 17 at day 0 and day 7. Seven days post boosting, spleen lymphocytes were isolated and labeled with CFSE and cultured in the presence of the same peptide for 4 and 8 days. Proliferated CD4⁺ and CD8⁺ lymphocytes in response to CD8 epitope were counted by a flow cytometry analysis using specific anti-CD4 and CD8 antibodies conjugated with FITC, respectively. Results were presented as cell dividing index (CDI), which is a ratio of a portion of CD4 or CD8 positive lymphocytes with low CFSE staining signal (CFSE^{low}) divided by a portion of high CFSE staining signal (CFSE^{high}) of cells.

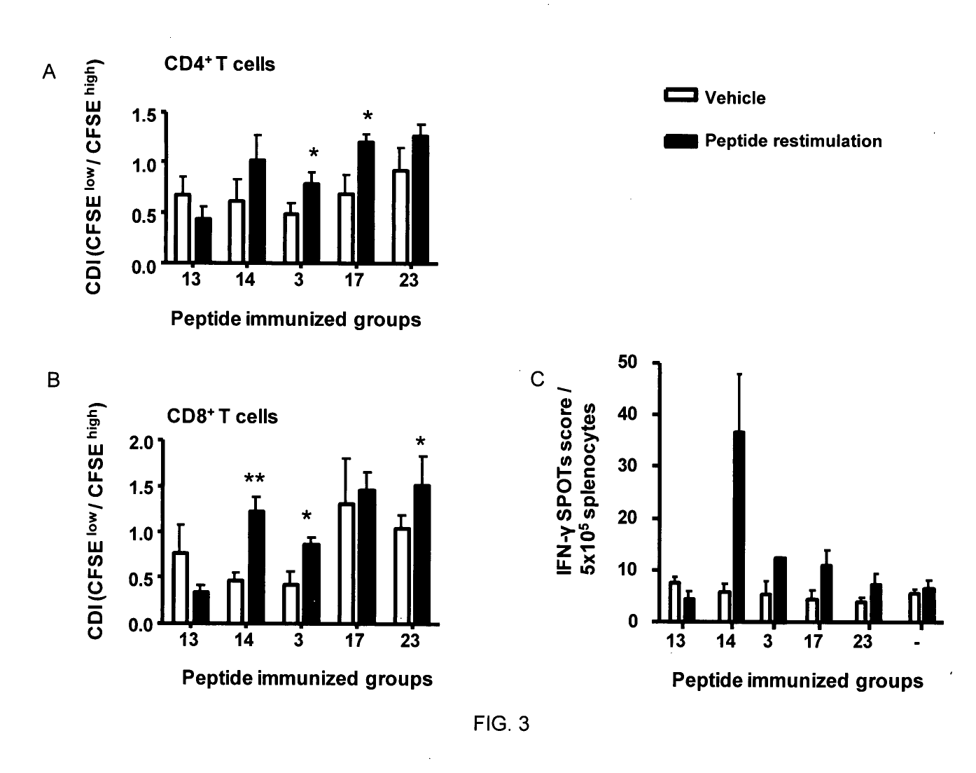

Neither CD4⁺ nor CD8⁺ lymphocyte proliferation in response to CD8 epitope were found at 4 days of peptide-splenocytes cultures (data not shown). CD4⁺ cells from peptide No. 13, 14, or 23-immunized mouse splenocytes did not manifest cell proliferation after 8 days of stimulation by the same peptide, as compared to the non-peptide stimulation. The splenocytes isolated from the peptide No. 3 or 17-immunized mice exhibited CD4⁺ cell proliferation (FIG. 3A). However, CD8⁺ population of lymphocytes from two doses of the peptide No. 14-, peptide No. 3-, or peptide No. 23-immunized mouse splenocytes showed activation of proliferation after 8 days of peptide'restimulation (FIG. 3B). Splenocytes from the peptide No. 13- and peptide No. 17-immunized mouse groups did not elicit CD8⁺ lymphocyte proliferation at the tested time intervals.

FIG. 3C shows the results of Th1 cytokines release using ELISPOT assays. Peptide-restimulated spleen lymphocytes (5×10⁶) were seeded onto 96-well plates pre-coated with anti-IFN-γ antibody to measure IFN-γ secretion using ELISPOT as described in Materials and Methods. *(p<0.05) and **(p<0.01) indicate a significant difference from the value obtained from the individual splenocytes without peptide restimulation. The lymphocytes from mice immunized with peptide No. 3, 13, 17, or 23 produced a background level of IFN-γ. In contrast, significantly higher levels of IFN-γ (36±13 spots/5×10⁵ splenocytes) were measured in the group of mice previously immunized with the peptide No. 14. In summary, CD8⁺ splenocytes activation from two doses of the peptide No. 14-immunized mice produced an allele-specific Th1 cytokine release.

### Induction of RSV F-Specific CTLs in Single Peptide-Vaccinated Mice

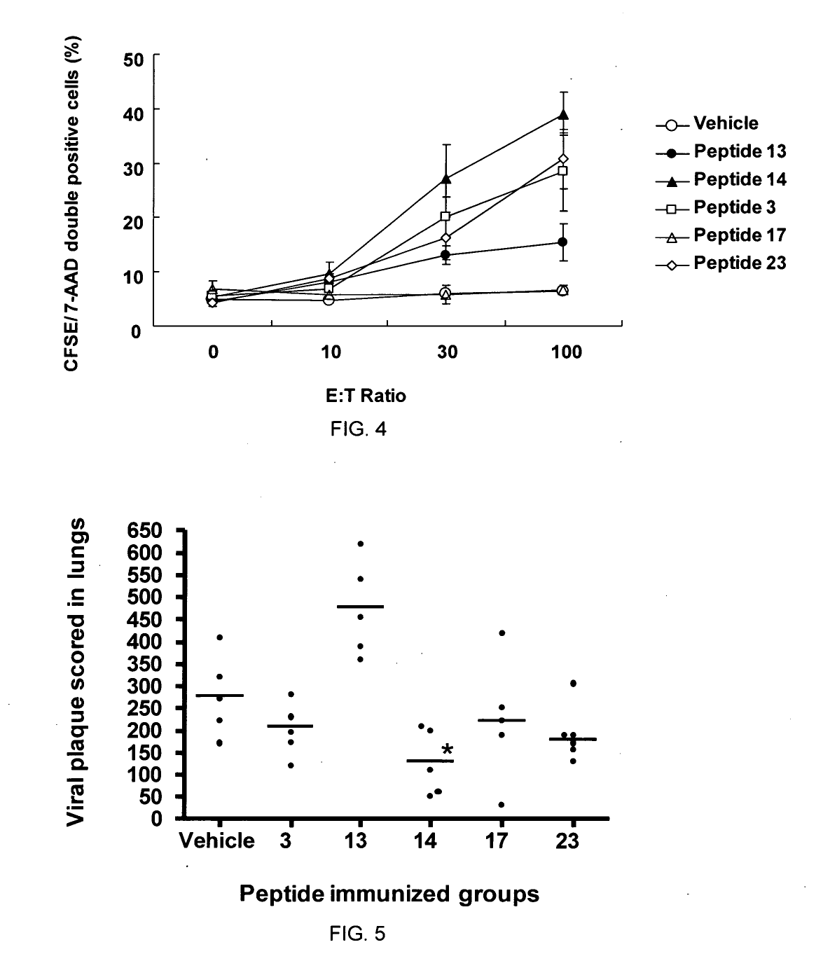

It has been reported that in primary RSV infection of BALB/c mice, CTLs generated against the F, matrix 2 (M2) and N viral proteins play a protective role in virus clearance. Our previous study has also shown that recombinant adenovirus carrying the RSV F gene can induce CTL activity that may result in the protection of RSV infection in mice. We investigated whether immunization of HLA-Tg B6 mice with these F protein peptides would lead to the induction of epitope-specific CTLs. Splenocytes isolated from peptide-immunized mice at two doses were stimulated with individual epitope peptides for 5 days. The peptide-stimulated splenocytes (effector) were then co-cultured with CFSE-labeled T2 (target) cells that had been previously loaded with the same peptide at the effector/target (E:T) ratios of 100:1, 300:1, and 10:1. The effector/target cell mixtures were stained with 7-AAD to detect the killed target cells (i.e., CFSE and 7-AAD double positive cells). A mean background percentage of the killed target cells co-cultured with the control splenocytes isolated from the mouse previously immunized with the vehicle IFA was scored (6.5% at the E:T ratio of 100:1). In contrast, RSV-infected mice mounted a significantly more vigorous response to peptides 3, 14, and 23 (28.3%, 38.9%, 30.7%, respectively) (FIG. 4). A moderate CTL response was observed in mice previously immunized with the peptide No. 13 (15.4%). No CTL response was detected in mice previously immunized with the peptide No. 17 (6.6%), which exhibited a low binding activity with the HLA-A*0201 molecule (MFI ratio=0.91) in T2-based binding assay. The results proved the immunopotency of peptide NOs. 14, 3 and 23 in CTL activation.

In FIG. 4, splenocytes prepared from mice previously immunized with the peptide Nos. 3, 13, 14, 17, 23, or vehicle were cultured and supplemented with murine IL-2 in the presence of 50.0 μg per mL of the same peptide for 5 days. Targets (T2 cells) were treated with 20 μg per mL of a synthetic peptide for 2 hours at 37° C. and labeled with CFSE prior to the assay. Un-loaded T2 served as a negative control. The cultured splenocytes were counted, and viable cells were co-cultured with 10⁴ peptide-preloaded T2 cells at a ratio of 100:1, 30:1, and 10:1 for four hours. The cell mixtures were labeled with 7-AAD and analyzed with a flow cytometry. Results were expressed as mean percentage of 7-AAD/CFSE positive population of cells ±two standard deviations for each experimental group (n=5).

### Protective Efficacy of CD8 Epitope-Based Vaccine Against Live RSV Challenge.

The immunoprotective ability of a single RSV F-derived CD8 epitope peptide against live RSV challenge was assessed in peptide-immunized mice. The parameters selected to assess protection included measurements of viremia in the lungs and the recovery from initial weight loss.

In FIG. 5, mice were immunized via the intranasal route twice with the immunogen as indicated before they were intranasally challenged with 10⁶ pfu of live RSV B1. Viral plaques in the lungs of the individual mice were determined 4 days post the challenge using plaque-forming assay as described in the methods. Results were expressed as the plaque number for each mouse. The horizontal bar represents the means of the plaque numbers obtained from each group. The symbol * (p<0.05) indicates being significantly different from the vehicle-immunized group. As shown in FIG. 5, a marked reduction in the number of lung viral plaques (140±65) was observed in animals that were immunized with two doses of the epitope peptide no. 14 and subsequently challenged with 1×10⁶ pfu of live RSV-B1 viruses, as compared to 282±116 plaques detected in the lung tissues of mice immunized with the adjuvant IFA alone. A slightly lower level of protection was observed in mice vaccinated with peptide no. 23. Animals immunized with peptide no. 3, 13, and 17 were not protected.

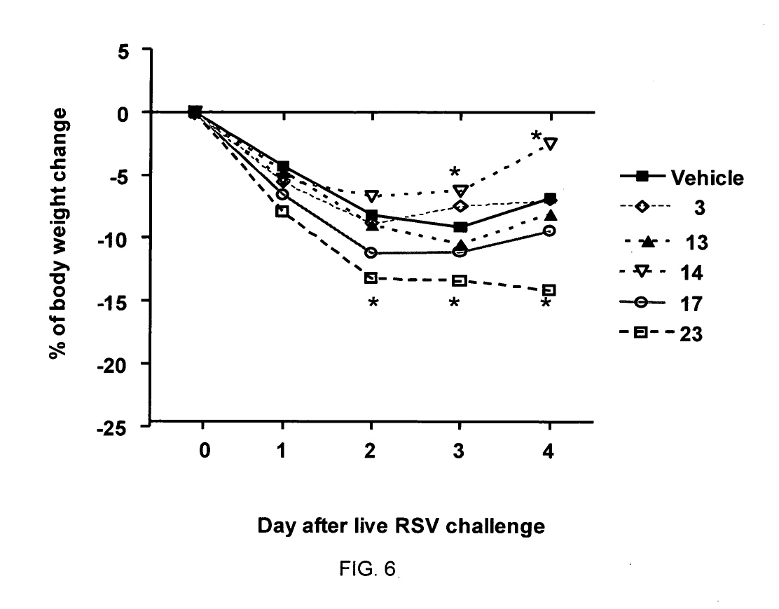

It has been reported that primary RSV infections of adult BALB/c mice causes an acute weight loss. FIG. 6 shows the results of monitoring the body weights of mice post live RSV-B 1 challenge. Mice were immunized twice via the intranasal route with vehicle, peptide Nos. 3, 13, 14, 17, or 23 as indicated. Each animal was subsequently intranasally inoculated with 10⁶ pfu of live RSV B1. The body weight of each mouse was recorded daily for 5 days post the viral challenge. Results were expressed as % (mean) (n=5). The symbol * (p<0.05) indicates there is a significant difference from the vehicle-immunized group. As shown in FIG. 6, a progressive body weight loss was found in the mice challenged with live RSV-B1 regardless of whether they had been immunized with individual CD8 epitope-derived peptide No. 3, 14, 17, or 23. In each case, a maximal body weight loss was reached on day 2 post the virus challenge. A significant recovery from the body weight loss was observed on days 3 and 4 in those having been immunized with the peptide No. 14 as compared to the vehicle (i.e., IFA alone)-immunized group (FIG. 6). The peptide No. 3 or 17-immunized mice showed no difference from the non-immunized mice, exhibiting mildly severe weight loss. The peptide No. 23-immunized mice showed the most severe body weight drop after the viral challenge. These results were in consistence with the previous study that RSV challenges in RSV F (vacvF)-expressing vaccinia virus-immunized mice may induce a weight loss and CD8⁺ T cells playing a direct role in mediating vaccine-enhanced diseases. The data here indicated that the identified HLA-A*0201 CD8 epitopes from RSV F possessed various degrees of immunogenicity in terms of induction of protective immunity against RSV infections and pathological effect in causing a weight reduction (peptide No. 23 vs. peptide No. 14).

### Discussion

CD8⁺ T cells recognize the processed small antigenic peptides (8-10 amino acids) in the context of class I MHC expressed by antigen-presenting cells (APCs), leading to a highly specific cytotoxicity to virus-infected cells. The F protein elicits CTL responses in human [26] and mice [22, 27] and stimulates an increased production of Th1 cytokines, IL-2, IL-12 and IFN-γ. Therefore, identification of CD8 epitopes derived from the F protein that are specifically subjected to human HLA is important in the study of peptide-based RSV vaccine development and vaccine-enhanced disease.

In the present study, we predicted human HLA-restricted CD8 epitopes from the F protein using a computational program for HLA Peptide Binding Predictions. These peptides have 9-mer amino acid core sequences and confirmed their association with the MHC class 1 molecule HLA-A*0201 in T2-based binding assay. These epitopes also can stimulate splenocytes from RSV or recombinant adenovirus carrying F gene-immunized HLA-A2.1 transgenic mice and lead to high IFN-γ and IL-2 production. The studies using transgenic mice vaccinated with a single peptide demonstrated that F273-281 (SEQ ID NO: 14) is able to induce CTL responses, reduce virus load in lung and decreases body weight loss in the mice challenged with live RSV. These novel HLA-A2-specific CD8 epitopes are characterized for the efficacy of protection against virus infection in the development of peptide-based RSV vaccine.

Primary antiviral CD8⁺ T-cell response is essential to the development of a host's adaptive immunity to RSV and other viral infections. During primary RSV infection, the F protein is a major target for CTL in mice and humans. Most studies focused on the identification of RSV F-specific immunodominant CD8 epitopes including a subdominant epitope F85-93 and two minor epitopes F92-106 and F249-258, which are mouse MHC-I (H-2K^{d})-restricted, and epitopes F118-126, F551-559 and F109-118, which are human-restricted HLA-B*57, HLA-Cw*12, and HLA-A*01, respectively. Yet no functional human HLA-A*02-restricted epitope had been identified. The remarkable finding of this study is that a series of human MHC-I HLA-A*0201 restricted CD8 epitopes spanning the RSV fusion (F) protein were identified by using in vitro cell culture screening of algorithmically synthetic HLA-A*0201/9-mer peptides for binding activity and comparative analysis of the Th1 cytokines responses raised from splenocytes co-cultured with individual peptides, in which the splenocytes were isolated from the HLA-A*0201 transgenic B6 mice infected with rAd-F0 or RSV-B1. Based on the characteristics of the higher binding activity with HLA molecules and the potency in the stimulation of Th1 cytokine (IFN-γ and IL-2) secretion from the splenocytes of vaccinated mice, respective RSV F CD8 epitopes (peptide Nos. 3, 13, 14 and 23) (FIG. 1 and FIG. 2) were selected and tested for their immunogenic properties as determined by the induction of cellular immune responses. Specific-peptide responsive CD8⁺ cells present in the splenocytes of the mice that were primed and boosted with a single epitope proliferated (FIG. 3) and activated CTL killing (FIG. 4).

The anti-viral immunity elicited by CD8 epitope-based vaccination conferred protections against live RSV-B1 viral challenge. This is evident from the significantly lower viral load recovered from the lungs of animals previously immunized subcutaneously with the IFA-emulsified peptide No. 14 but not with the peptide No. 3, 17, or 23 (FIG. 5). Vaccination with the peptide No. 14 allowed for an accelerated recovery from the initial body weight loss following live RSV challenge at day 3, which was faster than that observed in the mice previously immunized with vehicle (i.e., IFA alone). Vaccination of the peptide No. 3 or 17 elicited no significant or mildly severe body weight loss induced by the virus challenge. Interestingly, a controversial result was that mice immunized with peptides No. 23 showed more of a severe weight loss than the vehicle-immunized mice (FIG. 6). It has been reported that CD8⁺ T cells induced in the mice previously immunized with vaccinia virus carrying the F gene attribute to weight loss in the presence of Th1 cytokine secretion. However, the underlying mechanisms were unclear. Here, we first reported that the peptide No. 23 spanning residues 559 to 567 of the F protein (F559-567) may contribute to the RSV F mediated weight loss upon RSV challenge. In contrast, the peptide No. 14 (F273-281) reduced the weight loss in the RSV-infected Tg B6 mice in accompany with CD8⁺ T cell activation and IFN-γ secretion.

A recent study has generally questioned the role of CTLs in the pathogenesis of human RSV infection. Very few CD8⁺ T cells in the lung infiltrates were found in human infants infected with lethal RSV. Additionally, there was little evidence for T cell cytokines in nasal secretion and few CTL can be recovered by BAL from the infants with bronchiolitis. The pathological staining of CD4⁺ and CD8⁺ T cells in the lungs of the peptide-immunized mice infected with RSV showed that few infiltrated CD4⁺ and CD8⁺ T cells were found in the peptide No. 3, 14 or 17-immunized groups, but more infiltrated CD4⁺ and CD8⁺ T cells were observed in the peptide No. 23-immunized mice.

The observation of eosinophilia induced in the peptide-immunized mice challenged with RSV showed that none of the peptide-immunized animals exhibited an increase in eosinophile count in the peripheral blood or in the lung (data not shown). These data indicated that the epitope of the peptide No. 23 contributed to the RSV F-mediated weight loss upon RSV challenge, which resulted in severe CD8⁺ lymphocytic inflammation but not eosinophilia in the mouse lung tissues. Protection studies showed that the resistance to RSV challenge was greater in the HLA-A*0201 transgenic mice immunized with the epitope peptide No. 14, which mounted stronger CTL responses than the animals previously immunized with other tested peptides. These results demonstrated that the various HLA-restricted CD8⁺ T cell epitopes identified in our model elicited differential immunogenicities that participated in CD8⁺ T cell-mediated viral clearance or vaccine-enhanced diseases particularly in weight loss. Immunization with a single CTL epitope has previously been shown to induce protection against viral infection or tumor growth. In some cases, protective responses were only induced following immunization with CTL epitope linked to a T helper epitope or other carrier. Our unpublished result showed that immunization with the peptide No. 14 mixed with HCV T helper epitope does not enhance more protective responses in the reduction of viral load in the lung of the RSV-infected mouse.

In conclusion, the invention relates to two HLA-A*0201-restricted CD8 epitopes that elicited immunodominant properties of activation of CD8⁺ T lymphocytic proliferation and killing activity. The epitope peptide No. 14, spanning residues 273 to 281 of the RSV F protein, elicited protective immunity against the RSV challenge as determined by reducing the viral load in the lung tissue and the degree of weight loss in the absence of pulmonary eosinophilia. The peptide No. 23, spanning residues 559 to 567 of F, was identified as a “pathological epitope” of CD8⁺ T cells as it contributed to the F-mediated weight loss.

All of the references cited herein are incorporated by reference in their entirety.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. An isolated peptide comprising a human HLA-A2 restricted T-cell epitope derived from the fusion protein of respiratory syncytial virus (RSV-F), wherein the length of the peptide is no more than 10 amino acid residues, and wherein the peptide comprises a HLA-A2 binding motif having a binding affinity to a human HLA-A2 molecule on a cell.

2. The peptide of claim 1, wherein the peptide is a 9-mer selected from the group consisting of SEQ ID NOs: 3, 9, 14, 15, 16, 23 and 24.

3. The peptide of claim 1, wherein the peptide has a binding affinity to a human LHA-A2 at least 10% higher than that of the hepatitis C virus capsid peptide of SEQ ID NO: 27.

4. The peptide of claim 1, wherein the peptide possesses the activity of inducing IFN-γ release from an HLA-A2 expressing splenocyte having been previously exposed to RSV.

5. The peptide of claim 4, wherein the peptide is a 9-mer selected from the group consisting of SEQ ID NOs: 3, 11, 12, 13, 14, 18, 19, 20 and 23.

6. The peptide of claim 1, wherein the peptide possesses the activity of inducing IL-2 release from an HLA-A2 expressing splenocyte having been previously exposed to RSV.

7. The peptide of claim 1, wherein the peptide possesses mammalian immunogenicity eliciting CD4+ T cell proliferation.

8. The peptide of claim 7, wherein the peptide is a 9-mer selected from the group consisting of SEQ ID NOs: 3 and 17.

9. The peptide of claim 1, wherein the peptide possesses mammalian immunogenicity eliciting CD8+ T cell proliferation.

10. The peptide of claim 9, wherein the peptide is a 9-mer selected from the group consisting of SEQ ID NOs: 3 and 14.

11. The peptide of claim 1, wherein the peptide possesses mammalian immunogenicity eliciting CD8+ T cell proliferation and IFN-γ secretion.

12. The peptide of claim 11 wherein the peptide possesses mammalian immunogenicity inducing cytotoxic T cell killing responses.

13. The peptide of claim 12, wherein the peptide is a 9-mer selected from the group consisting of SEQ ID NOs: 3, 13, 23 and 14.

14. The peptide of claim 1, wherein the peptide possesses immunoprotective property against RSV infection and is devoid of the sequence of SEQ ID NO: 23.

15. A composition comprising a pharmacologically effective amount of one or more than one isolated peptide according to claim 14 and an adjuvant.

16. The composition of claim 15, wherein the adjuvant is at least one selected from the group consisting of AlPO₄, CFA/IFA, cholera toxin, Escherichia coli heat-labile enterotoxin (LT), liposome, immune-stimulating complex (ISCOM), and immunostimulatory sequences oligodeoxynucleotides (ISS-ODN).

17. A method of providing an immmnoprotective benefit to a human against RSV infection comprising the steps of:
administering a composition according to claim 15 to a human in need thereof, thereby providing an immmnoprotective benefit to the human against RSV infection.

18. The method of claim 17, wherein the composition comprises the peptide of SEQ ID NOs: 14.

19. The peptide of claim 1, wherein the peptide does not induce pulmonary eosinophilia in a mammal.

20. The peptide of claim 1, wherein the cell is an antigen presenting cell.
